(19) United States
(12) Patent Application Publication
(10) Pub. No.: US 2023/0077818 A1
(43) Pub. Date: Mar. 16, 2023

(54) METHOD FOR ESTABLISHING MEDICINE SYNERGISM PREDICTION MODEL, PREDICTION METHOD AND CORRESPONDING APPARATUS

(71) Applicant: BEIJING BAIDU NETCOM SCIENCE TECHNOLOGY CO., LTD., Beijing (CN)

(72) Inventors: Jing HU, Beijing (CN); Guodong ZHAO, Beijing (CN)

(73) Assignee: BEIJING BAIDU NETCOM SCIENCE TECHNOLOGY CO., LTD., Beijing (CN)

(21) Appl. No.: 17/844,103

(22) Filed: Jun. 20, 2022

(30) Foreign Application Priority Data
Sep. 13, 2021 (CN) 202111069600.X
Dec. 24, 2021 (CN) 202111597912.8

(51) Int. Cl. G06N 5/02; G16H 20/10; G16H 70/40; G16H 50/70

(52) U.S. Cl. CPC G06N 5/022; G16H 20/10; G16H 70/40; G16H 50/70

(57) ABSTRACT

The present disclosure discloses a method for establishing a medicine synergism prediction model, a prediction method and corresponding apparatus, and relates to deep learning and artificial intelligence (AI) medical technologies in the field of AI technologies. A specific implementation solution includes: acquiring a relation graph, nodes in the relation graph including medicine nodes and protein nodes, and edges indicating that interaction exists between the nodes; collecting, from the relation graph, a medicine node pair with definite synergism and a label of whether the medicine node pair has synergism as training samples; and training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph attention network.

### CROSS-REFERENCE TO RELATED APPLICATIONS

The present application claims the priority of Chinese Patent Application No. 202111069600.X, filed on Sep. 13, 2021, with the title of “METHOD FOR ESTABLISHING MEDICINE SYNERGISM PREDICTION MODEL, PREDICTION METHOD AND CORRESPONDING APPARATUS” and the priority of Chinese Patent Application No. 202111597912.8, filed on Dec. 24, 2021, with the title of “METHOD FOR ESTABLISHING MEDICINE SYNERGISM PREDICTION MODEL, PREDICTION METHOD AND CORRESPONDING APPARATUS.” The disclosures of the above applications are incorporated herein by reference in their entirety.

### FIELD OF THE DISCLOSURE

The present disclosure relates to the field of computer application technologies, and in particular, to deep learning and artificial intelligence (AI) medical technologies in the field of AI technologies.

### BACKGROUND OF THE DISCLOSURE

Combination medication refers to simultaneous or sequential application of two or more medicines for the purpose of treatment, mainly to increase the efficacy of the medicines or reduce toxic and side effects of the medicines. However, opposite results may also be produced. Therefore, rational combination medication is very important. The rational combination medication is based on medicine synergism. However, screening the medicine synergism from the experimental end consumes lots of manpower and material resources.

### SUMMARY OF THE DISCLOSURE

In view of the above, the present disclosure provides a method for establishing a medicine synergism prediction model, a prediction method and corresponding apparatus, so as to reduce labor and material costs.

According to a first aspect of the present disclosure, a method for establishing a medicine synergism prediction model is provided, including acquiring a relation graph, nodes in the relation graph including medicine nodes and protein nodes, and edges indicating that interaction exists between the nodes; collecting, from the relation graph, a medicine node pair with definite synergism and a label of whether the medicine node pair has synergism as training samples; and training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph attention network.

According to a second aspect of the present disclosure, a medicine synergism prediction method is provided, including determining a to-be-identified medicine node pair from a relation graph; and predicting the to-be-identified medicine node pair by using a medicine synergism prediction model, to obtain a prediction result indicating whether the to-be-identified medicine node pair has synergism; wherein the medicine synergism prediction model is pre-trained with the method as described above.

According to a third aspect of the present disclosure, an apparatus for establishing a medicine synergism prediction model is provided, including a graph acquisition unit configured to acquire a relation graph, nodes in the relation graph including medicine nodes and protein nodes, and edges indicating that interaction exists between the nodes; a sample collection unit configured to collect, from the relation graph, a medicine node pair with definite synergism and a label of whether the medicine node pair has synergism as training samples; and a model training unit configured to train the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph attention network.

According to a fourth aspect of the present disclosure, a medicine synergism prediction apparatus is provided, including a determination unit configured to determine a to-be-identified medicine node pair from a relation graph; and a prediction unit configured to predict the to-be-identified medicine node pair by using a medicine synergism prediction model, to obtain a prediction result indicating whether the to-be-identified medicine node pair has synergism; wherein the medicine synergism prediction model is pre-trained by the apparatus described above.

According to a fifth aspect of the present disclosure, an electronic device is provided, including at least one processor; and a memory in communication connection with the at least one processor; wherein the memory stores instructions executable by the at least one processor, and the instructions are executed by the at least one processor to enable the at least one processor to perform the method as described above.

According to a sixth aspect of the present disclosure, a non-transitory computer-readable storage medium storing computer instructions, wherein the computer instructions are configured to cause a computer to perform the method as described above.

According to a seventh aspect of the present disclosure, a computer program product is provided, including a computer program, wherein, when the computer program is executed by a processor, the method as described above is performed.

It should be understood that the content described in this part is neither intended to identify key or significant features of the embodiments of the present disclosure, nor intended to limit the scope of the present disclosure. Other features of the present disclosure will be made easier to understand through the following description.

### BRIEF DESCRIPTION OF DRAWINGS

The accompanying drawings are intended to provide a better understanding of the solutions and do not constitute a limitation on the present disclosure. In the drawings,

FIG. 1 is a flowchart of a method for establishing a medicine synergism prediction model according to an embodiment of the present disclosure;

FIG. 2 is a schematic relation graph according to an embodiment of the present disclosure;

FIG. 3 is a schematic diagram of the medicine synergism prediction model according to an embodiment of the present disclosure;

FIG. 4 is a flowchart of a medicine synergism prediction method according to an embodiment of the present disclosure;

FIG. 5 is a structural diagram of an apparatus for establishing a medicine synergism prediction model according to an embodiment of the present disclosure;

FIG. 6 is a structural diagram of a medicine synergism prediction apparatus according to an embodiment of the present disclosure; and

FIG. 7 is a block diagram of an electronic device configured to implement embodiments of the present disclosure.

### DETAILED DESCRIPTION OF PREFERRED EMBODIMENTS

Exemplary embodiments of the present disclosure are illustrated below with reference to the accompanying drawings, which include various details of the present disclosure to facilitate understanding and should be considered only as exemplary. Therefore, those of ordinary skill in the art should be aware that various changes and modifications can be made to the embodiments described herein without departing from the scope and spirit of the present disclosure. Similarly, for clarity and simplicity, descriptions of well-known functions and structures are omitted in the following description.

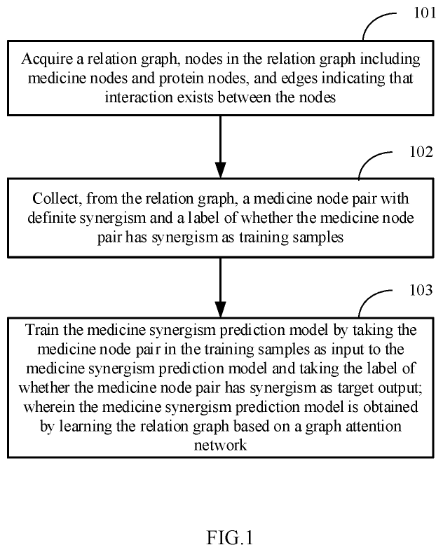

The present disclosure provides a manner of establishing a medicine synergism prediction model and a prediction manner based on the model. FIG. 1 is a flowchart of a method for establishing a medicine synergism prediction model according to an embodiment of the present disclosure. The method is performed by an apparatus for establishing a medicine synergism prediction model. The apparatus may be an application located in a computer terminal or a functional unit in an application located in a computer terminal such as a plug-in or a Software Development Kit (SDK), or located on a server side, which is not particularly limited herein in the embodiment of the present invention. As shown in FIG. 1, the method may include the following steps.

In 101, a relation graph is acquired, nodes in the relation graph including medicine nodes and protein nodes, and edges indicating that interaction exists between the nodes.

In 102, a medicine node pair with definite synergism and a label of whether the medicine node pair has synergism are collected from the relation graph as training samples.

In 103, the medicine synergism prediction model is trained by taking the medicine node pair in the training samples as input to the medicine synergism prediction model and taking the label of whether the medicine node pair has synergism as target output; wherein the medicine synergism prediction model is obtained by learning the relation graph based on a graph attention network.

As can be seen, in the present disclosure, the medicine synergism prediction model is obtained by learning interaction between the medicine node pair with definite synergism in the relation graph in a preset neighborhood range based on the graph attention network. Based on this, automatic prediction of medicine synergism can be realized, which saves more labor and material costs than all trials.

The above steps are described in detail below with reference to embodiments. Firstly, step 101 is described in detail.

Medicines mainly act on animals (including humans), and distribution, transport, metabolism and efficacy of the medicines in the animals are all related to protein. Therefore, the study on medicine effects mainly focuses on the interaction between medicines and protein, and the protein on which the medicines act are generally called target protein. That is, the interaction between the medicines and the protein may be acquired from previous experimental data.

The interaction between the protein is also a relatively mature technology, which has accumulated a large number of experimental data and is in the process of continuous development. The interaction between the protein can better annotate protein functions and decode life phenomena, and is particularly useful in medicine design.

Part of medicine node pairs with definite synergism have been obtained through experiments in the early stage, and this part of experimental data can also be acquired and used for model training in the present disclosure.

A relation graph may be constructed by using data of the interaction between medicines and protein, between protein and between medicines. The relation graph consists of nodes and edges. The nodes include medicines and protein. Edges between the nodes indicate that interaction exists between the nodes. The interaction between the medicines includes synergism. That is, if synergism exists between the medicines, edges exist between corresponding medicine nodes in the relation graph. Otherwise, no edges exist.

In the early stage, the experiment relies on a carrier, namely tissue (the “tissue” referred to in the embodiment of the present disclosure refers to biological tissue). For example, the experiment is conducted in a cell line, organ, tumor tissue or the like. Therefore, as a preferred implementation, the relation graph in the present disclosure is a relation graph for particular tissue, such as a relation graph for lung cancer or a relation graph for thyroid cancer. In this case, the edges in the relation graph indicate that the interaction exists between the nodes on the particular tissue, and the relation graph is actually a protein interaction network reflecting tissue specificity. The relation graph can save more computational power and reflect characteristics of the tissue specificity, so that a subsequently established model has stronger prediction capability and reduces the possibility of model overfitting.

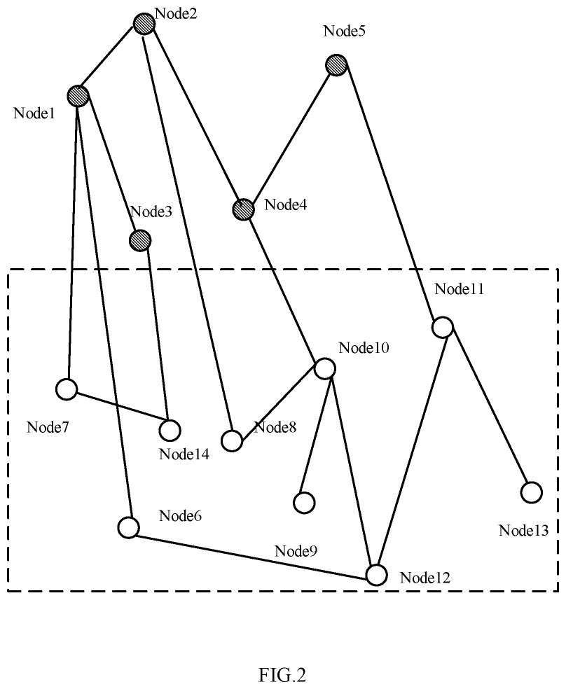

FIG. 2 is a schematic relation graph according to an embodiment of the present disclosure. In the figure, solid nodes 1-5 represent medicine nodes, and hollow nodes 6-14 represent protein nodes. Edges between the medicine nodes and the protein nodes represent interaction between medicines and protein, and edges between the protein nodes represent interaction between the corresponding protein. Edges between the medicine nodes indicate that synergism exists between the medicine nodes. In the relation graph, a part marked by the dotted line is a lung-cancer-specific protein interaction network. Correspondingly, the medicine node pairs with synergism (i.e., edges) are also taken only from experimental results in lung cancer cell lines.

In the relation graph, edge connections exist between the medicine node pairs with definite synergism, and no edge exists between some medicine nodes with indefinite synergism or definitely without synergism. In step 102 of the above embodiment, a medicine node pair with definite synergism and a label of whether the medicine node pair has synergism may be collected from the relation graph as a training sample.

Taking FIG. 2 as an example, definite synergism exists between Medicine Node 1 and Medicine Node 2. Therefore, Medicine Node 1 and Medicine Node 2 may be collected as a medicine node pair to form a training sample, for example,

training sample 1: (Medicine Node 1-Medicine Node 2, with synergism in lung cancer cell lines);

training sample 2: (Medicine Node 1-Medicine Node 2, with synergism in lung cancer cell lines).

Synergism definitely does not exist between Medicine Node 2 and Medicine Node 5. Therefore, Medicine Node 2 and Medicine Node 5 may be collected as a medicine node pair to form a training sample, for example,

training sample 3: (Medicine Node 2-Medicine Node 5, with no synergism in lung cancer cell lines).

Step 103 “training the medicine synergism prediction model by taking the medicine node pair in the training samples as input to the medicine synergism prediction model and taking the label of whether the medicine node pair has synergism as target output” is described in detail below with reference to embodiments.

In the present disclosure, during the training of the medicine synergism prediction model, connection relations between the medicine nodes in the training samples reflected in the relation graph are learned based on a graph convolutional network and an attention mechanism. In addition to relations between the medicine nodes, the connection relations further include relations between the medicine nodes and the protein as well as relations between the protein.

Since the relation graph in the embodiment of the present disclosure is a relation graph for particular tissue, an amount of calculation is reduced and the training efficiency can be significantly improved.

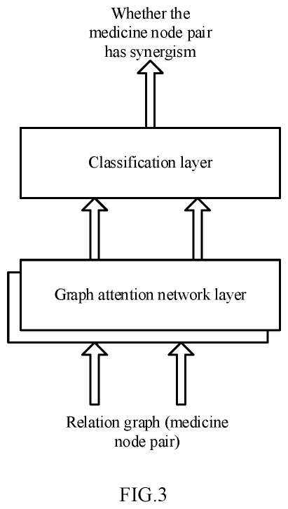

FIG. 3 is a schematic structural diagram of the medicine synergism prediction model according to an embodiment of the present disclosure. As shown in FIG. 3, the medicine synergism prediction model may mainly include a graph attention network layer and a classification layer. The graph attention network layer may include more than one graph convolutional network layer.

After the medicine nodes in the training samples are inputted to the medicine synergism prediction model, the graph attention network layer is configured to perform Attention by using features of the medicine nodes in the relation graph and features of neighbor nodes of the medicine nodes, to obtain vector representations of the medicine nodes.

As a preferred implementation, the graph attention network layer may embed the features of the medicine nodes and the features of the neighbor nodes of the medicine nodes and perform Attention on feature vectors of the nodes obtained by embedding, to obtain the vector representations of the medicine nodes.

That is, the vector representations of the medicine nodes after Attention are not only determined by the feature vectors of the medicine nodes but also affected by the neighbor nodes. A vector representation h′ᵢ of a medicine node may be determined by the following formula:

$${h}_{i}^{′} = σ ( {\sum }_{j ∈ {N}_{i}} {α}_{ij} W {h}_{j} ) \qquad ( 1 )$$

where σ( ) denotes a nonlinear function, Nⱼ represents a set consisting of the medicine node and all neighbor nodes thereof, W denotes a parameter matrix of the graph convolutional network layer, belonging to to-be-updated model parameters, and denotes an attention weight.

$${α}_{ij} = \frac{exp ( Leaky Relu ( {a}^{T} [ W {h}_{i} ∥ W {h}_{j} ] )}{{\sum }_{k ∈ {N}_{i}} exp ( Leaky Relu ( {a}^{T} [ W {h}_{i} ∥ W {h}_{k} ] )} \qquad ( 2 )$$

where a denotes an attention matrix consisting of attention weights in previous iteration, and Leaky Relu( ) denotes a linear unit activation function with leakage correction adopted in a graph attention network layer, which may be replaced by other activation functions. ∥ denotes an operation of splicing two vectors.

After the processing of the attention mechanism, the graph attention network may scale features through a fully connected layer, Relu is taken as a non-linear function between linear layers, and output of the last layer may take Sigmoid as a nonlinear function.

Taking the medicine node pair consisting of Node 1 and Node 2 in FIG. 2 as an example, neighbor nodes of Node 1, namely Node 2, Node 3, Node 6 and Node 7, are sampled, features of Nodes 1, 2, 3, 6 and 7 are embedded respectively to obtain feature vectors of the nodes, and then Attention is performed according to the formula (2) and the formula (1) to obtain a vector representation of Node 1.

Neighbor nodes of Node 2, namely Node 1, Node 4 and Node 8, are sampled, features of Nodes 2, 1, 4 and 8 are embedded to obtain feature vectors of the nodes, and then Attention is performed according to the formula (2) and the formula (1) to obtain a vector representation of Node 2.

The medicine synergism prediction model adopts the attention mechanism to perform feature iteration, which is faster and can take into account direct correlation between different medicines, enhancing the robustness and generalization of the model.

In addition, the graph attention network layer may also perform processing with multi-head attention. When a multi-head attention mechanism is adopted, multi-head attention processing is actually performed by using features of the medicine nodes in the relation graph and features of the neighbor nodes of the medicine nodes, and then a plurality of vector representations obtained by the multi-head attention processing, to obtain the vector representations of the medicine nodes. In this case, a vector representation h′ᵢ of a medicine node may be determined by the following formula:

$${h}_{i}^{′} = {}_{k = 1}^{K} ∥ σ ( {\sum }_{j ∈ {N}_{i}} {α}_{ij} W {h}_{j} ) \qquad ( 3 )$$

where K denotes a number of attention heads.

During the embedding, the features of the nodes used may include molecular weight, molecular activity, etc. In the embodiment of the present disclosure, initial values used by the medicine synergism prediction model during the embedding may be either preset values or pre-trained by a compound pre-training task. Compounds including medicines and protein are pre-collected as training samples, and some groups are randomly masked in the training samples. A Masked Language Model (MLM) may be used for the compound pre-training task. The MLM includes an embedding unit and a prediction unit. Training samples of some masked groups are inputted to the MLM. The embedding unit extracts the feature vectors of the atoms in the training samples, namely the compound. The prediction unit predicts masked content according to the feature vectors of the atoms. A training objective is to minimize a difference between a prediction result of the prediction unit and content of the some masked groups in the training samples. After the training, a pre-training model is constructed by using the embedding unit obtained by training. The constructed pre-training model may include an embedding unit and an integration unit. The feature vectors of the atoms in the nodes may be obtained by processing the nodes used in the present disclosure through the embedding unit in the pre-training model, and then the feature vectors of the atoms are integrated by the integration unit to obtain the feature vectors of the nodes, namely the entire compound. The feature vectors of the nodes may be used as initial values of the feature vectors of the nodes to train the medicine synergism prediction model. The integration performed by the integration unit may be splicing, pooling or the like.

Still referring to FIG. 3, the classification layer is configured to obtain, by using the vector representations corresponding to the medicine nodes in the medicine node pair, a classification result indicating whether the medicine node pair has synergism. A training objective of the medicine synergism prediction model is to minimize a difference between the classification result and the corresponding label.

Since the constructed relation graph is a relation graph for particular tissue, the classification layer may splice the vector representations of the medicine nodes in the medicine node pair with a vector representation of the tissue, and perform classification through a linear layer (such as Softmax layer) by using a vector representation obtained by splicing, to obtain the classification result indicating whether the medicine node pair has synergism.

Still taking FIG. 2 as an example, after the vector representation of Node 1 and the vector representation of Node 2 are obtained in the above process, the classification layer splices the vector representation corresponding to Node 1 and the vector representation corresponding to Node 2 with a vector representation of lung tissue, and then performs classification by using a vector representation obtained by splicing. The classification result is whether the medicine node pair has synergism. In an actual classification process, a probability value of existence of synergism between Node 1 and Node 2 is actually obtained.

As can be seen, the medicine synergism prediction model also considers characterization of the particular tissue during classification prediction, so that the established model has better discrimination capability.

During the above training, a loss function may be constructed by using the training objective. In each iteration, model parameters are updated with the value of the loss function until a preset training end condition is reached. The training end condition may be such as convergence of the loss function or a number of iterations reaching a preset number threshold.

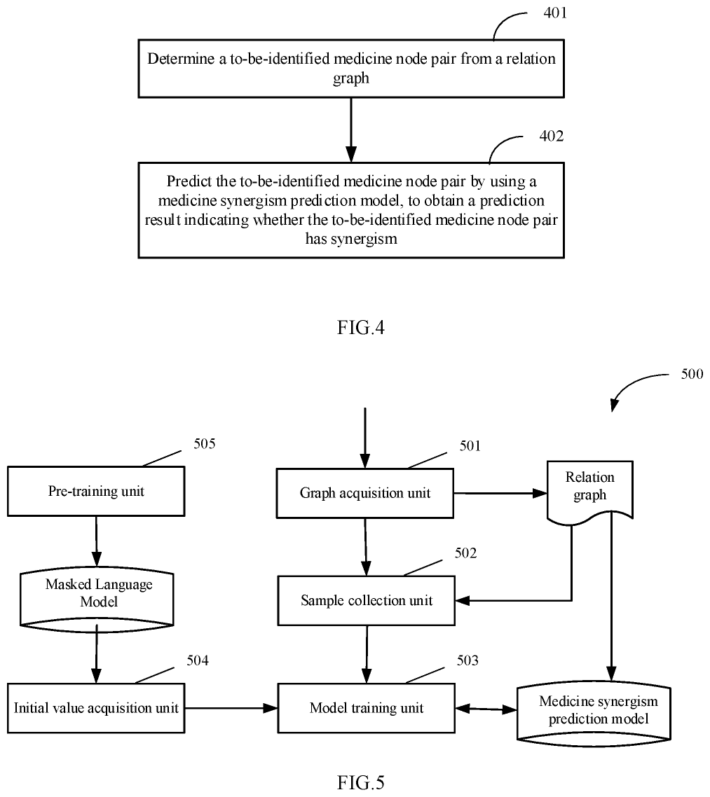

FIG. 4 is a flowchart of a medicine synergism prediction method according to an embodiment of the present disclosure. The method is performed by a medicine synergism prediction apparatus. The apparatus may be an application located in a computer terminal or a functional unit in an application located in a computer terminal such as a plug-in or an SDK, or located on a server side, which is not particularly limited herein in the embodiment of the present invention. As shown in FIG. 4, the method may include the following steps.

In 401, a to-be-identified medicine node pair is determined from a relation graph.

In the embodiment of the present disclosure, a medicine node pair with indefinite synergism in the relation graph may be predicted. That is, the medicine node pair with indefinite synergism in the relation graph may be taken as the to-be-identified medicine node pair.

For a newly generated medicine, the interaction between the new medicine and protein has to be verified by experiments, so it may be acquired through experimental data. In this case, the new medicine may be added to the relation graph to predict synergism with other medicine nodes. That is, the new medicine and other medicine nodes in the relation graph may form to-be-identified medicine node pairs respectively.

In 402, the to-be-identified medicine node pair is predicted by using a medicine synergism prediction model, to obtain a prediction result indicating whether the to-be-identified medicine node pair has synergism.

After the to-be-identified medicine node pair is inputted to the medicine synergism prediction model, the graph attention network layer of the medicine synergism prediction model performs Attention by using features of medicine nodes in the to-be-identified medicine node pair in the relation graph and features of neighbor nodes of the medicine nodes, to obtain vector representations of the medicine nodes. Then, the classification layer obtains, by using the vector representations of the medicine nodes in the medicine node pair, a classification result indicating whether the medicine node pair has synergism. The classification result is a prediction result.

As a specific implementation, the graph attention network layer may perform multi-head attention processing by using features of the medicine nodes in the relation graph and features of the neighbor nodes of the medicine nodes, and merge a plurality of vector representations obtained by the multi-head attention processing, to obtain the vector representations of the medicine nodes.

In addition, since the relation graph in the present disclosure is a relation graph for particular tissue, as a preferred implementation, the classification layer may splice the vector representations of the medicine nodes in the to-be-identified medicine node pair with a vector representation of the particular tissue, and perform classification by using a vector representation obtained by splicing, to obtain the classification result indicating whether the to-be-identified medicine node pair has synergism.

The above is a detailed description of the method according to the present disclosure, and the following is a detailed description of the apparatus according to the present disclosure with reference to embodiments.

FIG. 5 is a structural diagram of an apparatus for establishing a medicine synergism prediction model according to an embodiment of the present disclosure. As shown in FIG. 5, the apparatus 500 may include: a graph acquisition unit 501, a sample collection unit 502 and a model training unit 503, and may further include an initial value acquisition unit 504 and a pre-training unit 505. Main functions of the component units are as follows.

The graph acquisition unit 501 is configured to acquire a relation graph, nodes in the relation graph including medicine nodes and protein nodes, and edges indicating that interaction exists between the nodes.

The sample collection unit 502 is configured to collect, from the relation graph, a medicine node pair with definite synergism and a label of whether the medicine node pair has synergism as training samples.

The model training unit 503 is configured to train the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph attention network.

As a preferred implementation, the relation graph is a relation graph for particular tissue and the edges in the relation graph indicate that interaction exists between the nodes on the particular tissue.

The medicine synergism prediction model includes a graph attention network layer and a classification layer.

The graph attention network layer is configured to perform Attention by using feature vectors of the medicine nodes in the relation graph and feature vectors of neighbor nodes of the medicine nodes, to obtain vector representations of the medicine nodes.

The classification layer is configured to obtain, by using the vector representations of the medicine nodes in the medicine node pair, a classification result indicating whether the medicine node pair has synergism.

A training objective of the model training unit is to minimize a difference between the classification result and the corresponding label.

Initial values of the feature vectors of the nodes may also be preset values. However, as a preferred implementation, the initial values of the feature vectors of the nodes may be pre-obtained from a compound pre-training task. That is, the initial value acquisition unit 504 obtains feature vectors of atoms in the nodes by using a pre-trained embedding unit; and integrates the feature vectors of the atoms by an integration unit, to obtain the feature vectors of the nodes as initial values of the feature vectors of the nodes used by the graph attention network.

Furthermore, the embedding unit may be pre-trained by the pre-training unit 505. Specifically, the pre-training unit 505 acquires a compound as a training sample; and trains an MLM including an embedding unit and a prediction unit by taking training samples of some randomly masked groups as input to the MLM.

The embedding unit extracts the feature vectors of the atoms in the training sample, the prediction unit predicts masked content according to the feature vectors of the atoms, and a training objective is to minimize a difference between a prediction result of the prediction unit and content of the some masked groups in the training sample.

The graph attention network layer may be specifically configured to perform multi-head attention processing by using features of the medicine nodes in the relation graph and features of the neighbor nodes of the medicine nodes; and merge a plurality of vector representations obtained by the multi-head attention processing, to obtain the vector representations of the medicine nodes.

As a preferred implementation, the classification layer may be configured to splice the vector representations of the medicine nodes in the medicine node pair with a vector representation of the particular tissue; and perform classification by using a vector representation obtained by splicing, to obtain the classification result indicating whether the medicine node pair has synergism.

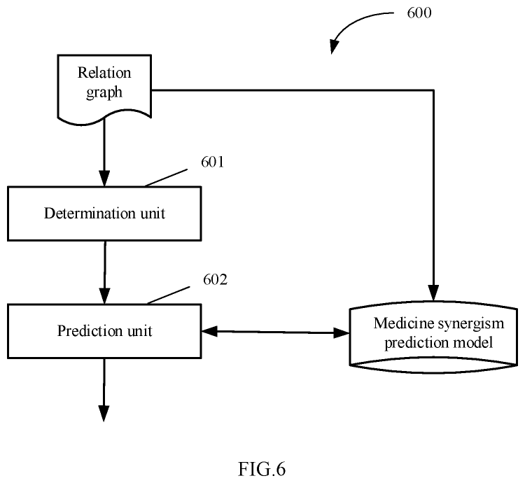

FIG. 6 is a structural diagram of a medicine synergism prediction apparatus according to an embodiment of the present disclosure. As shown in FIG. 6, the apparatus 600 may include: a determination unit 601 and a prediction unit 602. Main functions of the component units are as follows.

The determination unit 601 is configured to determine a to-be-identified medicine node pair from a relation graph.

In the embodiment of the present disclosure, a medicine node pair with indefinite synergism in the relation graph may be predicted. That is, the determination unit 601 may take the medicine node pair with indefinite synergism in the relation graph as the to-be-identified medicine node pair.

For a newly generated medicine, the interaction between the new medicine and protein has to be verified by experiments, so it may be acquired through experimental data. In this case, the new medicine may be added to the relation graph to predict synergism with other medicine nodes. That is, the determination unit 601 may form to-be-identified medicine node pairs respectively according to the new medicine and other medicine nodes in the relation graph.

The prediction unit 602 is configured to predict the to-be-identified medicine node pair by using a medicine synergism prediction model, to obtain a prediction result indicating whether the to-be-identified medicine node pair has synergism.

The medicine synergism prediction model is pre-trained by the apparatus shown in FIG. 5.

After the to-be-identified medicine node pair is inputted to the medicine synergism prediction model, the graph attention network layer of the medicine synergism prediction model performs Attention by using features of medicine nodes in the to-be-identified medicine node pair in the relation graph and features of neighbor nodes of the medicine nodes, to obtain vector representations of the medicine nodes. Then, the classification layer obtains, by using the vector representations of the medicine nodes in the medicine node pair, a classification result indicating whether the medicine node pair has synergism. The classification result is a prediction result.

As a specific implementation, the graph attention network layer may perform multi-head attention processing by using features of the medicine nodes in the relation graph and features of the neighbor nodes of the medicine nodes, and merge a plurality of vector representations obtained by the multi-head attention processing, to obtain the vector representations of the medicine nodes.

In addition, since the relation graph in the present disclosure is a relation graph for particular tissue, as a preferred implementation, the classification layer may splice the vector representations of the medicine nodes in the to-be-identified medicine node pair with a vector representation of the particular tissue, and perform classification by using a vector representation obtained by splicing, to obtain the classification result indicating whether the to-be-identified medicine node pair has synergism.

Tissue-specific relation graphs for prostate, large intestine, ovary, skin and lung are constructed respectively by using the manner according to the above embodiment of the present disclosure as training sets. Thymus tissue is selected as a training set. After the training and testing, related indicator results are shown in the table below.

TABLE 1

| Prediction method | ROC AUC | PR AUC | Recall rate |
|---|---|---|---|
| Conventional method | 0.53 ± 0.006 | 0.392 ± 0.03 | 0.078 ± 0.01 |
| Graph attention mechanism | 0.58 ± 0.02 | 0.244 ± 0.05 | 1.0 ± 0.0 |
| combined with random | | | |
| initialization | | | |
| Graph attention mechanism | 0.67 ± 0.04 | 0.40 ± 0.02 | 1.0 ± 0.0 |
| combined with pre-trained | | | |
| node feature | | | |

The conventional method mentioned in Table 1 is to extract feature vectors of medicines to construct feature vectors of medicine pairs, and then perform modeling, training and prediction by using a fully connected neural network. A method of combining a graph attention mechanism with random initialization means adopting the graph attention mechanism according to the embodiment of the present disclosure, but training the feature vectors of the nodes by random initialization. A method of combining a graph attention mechanism with pre-trained node features means adopting the graph attention mechanism according to the embodiment of the present disclosure, but pre-training initial values of the feature vectors of the nodes by a CCI task. Receiver Operating Characteristic Area Under the Curve (ROC AUC) and Precision-Recall Area Under the Curve (PR AUC) both reflect the model performance, and the closer they are to 1, the higher the model accuracy.

As can be seen from Table 1, the convention method has poor generalization performance, with poor performance and an extremely low recall rate in samples that have not been trained by algorithms, indicating that most medicine node pairs with synergism are determined to have no synergism.

The method of combining a graph attention mechanism with random initialization has poor PR AUC, but ROC AUC and the recall rate both higher than those in the conventional method. The method of combining a graph attention mechanism with pre-trained node features is better than the conventional method in the ROC AUC, the PR AUC and the recall rate, and the recall rate reaches 100%. It shows that the algorithm framework according to the present disclosure may indeed observe some similar characteristics from medicine features and medicine-protein interaction features based on the attention mechanism, so as to predict the mechanism of medicine synergism. It also shows that upstream pre-training of features of medicine nodes and protein nodes can significantly enhance the prediction capability of the model.

Various embodiments in the specification are described progressively. Same and similar parts among the embodiments may be referred to one another, and each embodiment focuses on differences from other embodiments. In particular, the apparatus embodiments are basically similar to the method embodiments, so the description thereof is relatively simple. Related parts may be obtained with reference to the corresponding description in the method embodiments.

Acquisition, storage and application of users' personal information involved in the technical solutions of the present disclosure comply with relevant laws and regulations, and do not violate public order and moral.

According to embodiments of the present disclosure, the present application further provides an electronic device, a readable storage medium and a computer program product.

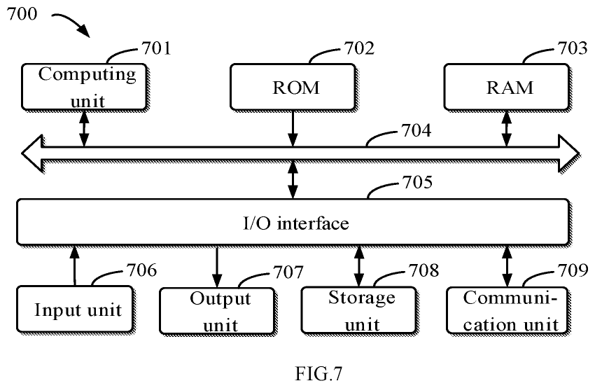

FIG. 7 is a block diagram of an electronic device configured to perform a method for establishing a medicine synergism prediction model and a prediction method according to embodiments of the present disclosure. The electronic device is intended to represent various forms of digital computers, such as laptops, desktops, workbenches, personal digital assistants, servers, blade servers, mainframe computers and other suitable computing devices. The electronic device may further represent various forms of mobile devices, such as personal digital assistants, cellular phones, smart phones, wearable devices and other similar computing devices. The components, their connections and relationships, and their functions shown herein are examples only, and are not intended to limit the implementation of the present disclosure as described and/or required herein.

As shown in FIG. 7, the device 700 includes a computing unit 701, which may perform various suitable actions and processing according to a computer program stored in a read-only memory (ROM) 702 or a computer program loaded from a storage unit 708 into a random access memory (RAM) 703. The RAM 703 may also store various programs and data required to operate the device 700. The computing unit 701, the ROM 702 and the RAM 703 are connected to one another by a bus 704. An input/output (I/O) interface 705 may also be connected to the bus 704.

A plurality of components in the device 700 are connected to the I/O interface 705, including an input unit 706, such as a keyboard and a mouse; an output unit 707, such as various displays and speakers; a storage unit 708, such as disks and discs; and a communication unit 709, such as a network card, a modem and a wireless communication transceiver. The communication unit 709 allows the device 700 to exchange information/data with other devices over computer networks such as the Internet and/or various telecommunications networks.

The computing unit 701 may be a variety of general-purpose and/or special-purpose processing components with processing and computing capabilities. Some examples of the computing unit 701 include, but are not limited to, a central processing unit (CPU), a graphics processing unit (GPU), various artificial intelligence (AI) computing chips, various computing units that run machine learning model algorithms, a digital signal processor (DSP), and any appropriate processor, controller or microcontroller, etc. The computing unit 701 performs the methods and processing described above, such as the method for establishing a medicine synergism prediction model and the prediction method. For example, in some embodiments, the method for establishing a medicine synergism prediction model and the prediction method may be implemented as a computer software program that is tangibly embodied in a machine-readable medium, such as the storage unit 708.

In some embodiments, part or all of a computer program may be loaded and/or installed on the device 700 via the ROM 702 and/or the communication unit 709. One or more steps of the method for establishing a medicine synergism prediction model and the prediction method described above may be performed when the computer program is loaded into the RAM 703 and executed by the computing unit 701. Alternatively, in other embodiments, the computing unit 701 may be configured to perform the method for establishing a medicine synergism prediction model and the prediction method by any other appropriate means (for example, by means of firmware).

Various implementations of the systems and technologies disclosed herein can be realized in a digital electronic circuit system, an integrated circuit system, a field programmable gate array (FPGA), an application-specific integrated circuit (ASIC), an application-specific standard product (ASSP), a system on chip (SOC), a complex programmable logic device (CPLD), computer hardware, firmware, software, and/or combinations thereof. Such implementations may include implementation in one or more computer programs that are executable and/or interpretable on a programmable system including at least one programmable processor, which can be special or general purpose, configured to receive data and instructions from a storage system, at least one input apparatus, and at least one output apparatus, and to transmit data and instructions to the storage system, the at least one input apparatus, and the at least one output apparatus.

Program codes configured to implement the methods in the present disclosure may be written in any combination of one or more programming languages. Such program codes may be supplied to a processor or controller of a general-purpose computer, a special-purpose computer, or another programmable data processing apparatus to enable the function/operation specified in the flowchart and/or block diagram to be implemented when the program codes are executed by the processor or controller. The program codes may be executed entirely on a machine, partially on a machine, partially on a machine and partially on a remote machine as a stand-alone package, or entirely on a remote machine or a server.

In the context of the present disclosure, machine-readable media may be tangible media which may include or store programs for use by or in conjunction with an instruction execution system, apparatus or device. The machine-readable media may be machine-readable signal media or machine-readable storage media. The machine-readable media may include, but are not limited to, electronic, magnetic, optical, electromagnetic, infrared, or semiconductor systems, apparatus or devices, or any suitable combinations thereof. More specific examples of machine-readable storage media may include electrical connections based on one or more wires, a portable computer disk, a hard disk, an RAM, an ROM, an erasable programmable read only memory (EPROM or flash memory), an optical fiber, a compact disk read only memory (CD-ROM), an optical storage device, a magnetic storage device, or any suitable combination thereof.

To provide interaction with a user, the systems and technologies described here can be implemented on a computer. The computer has: a display apparatus (e.g., a cathode-ray tube (CRT) or a liquid crystal display (LCD) monitor) for displaying information to the user; and a keyboard and a pointing apparatus (e.g., a mouse or trackball) through which the user may provide input for the computer. Other kinds of apparatus may also be configured to provide interaction with the user. For example, a feedback provided for the user may be any form of sensory feedback (e.g., visual, auditory, or tactile feedback); and input from the user may be received in any form (including sound input, voice input, or tactile input).

The systems and technologies described herein can be implemented in a computing system including background components (e.g., as a data server), or a computing system including middleware components (e.g., an application server), or a computing system including front-end components (e.g., a user computer with a graphical user interface or web browser through which the user can interact with the implementation schema of the systems and technologies described here), or a computing system including any combination of such background components, middleware components or front-end components. The components of the system can be connected to each other through any form or medium of digital data communication (e.g., a communication network). Examples of the communication network include: a local area network (LAN), a wide area network (WAN) and the Internet.

The computer system may include a client and a server. The client and the server are generally far away from each other and generally interact via the communication network. A relationship between the client and the server is generated through computer programs that run on a corresponding computer and have a client-server relationship with each other. The server may be a cloud server, also known as a cloud computing server or cloud host, which is a host product in the cloud computing service system to solve the problems of difficult management and weak business scalability in the traditional physical host and a virtual private server (VPS). The server may also be a distributed system server, or a server combined with blockchain.

It should be understood that the steps can be reordered, added, or deleted using the various forms of processes shown above. For example, the steps described in the present application may be executed in parallel or sequentially or in different sequences, provided that desired results of the technical solutions disclosed in the present disclosure are achieved, which is not limited herein.

The above specific implementations do not limit the protection scope of the present disclosure. Those skilled in the art should understand that various modifications, combinations, sub-combinations, and replacements can be made according to design requirements and other factors. Any modifications, equivalent substitutions and improvements made within the spirit and principle of the present disclosure all should be included in the protection scope of the present disclosure.

What is claimed is:

1. A method for establishing a medicine synergism prediction model, comprising:
acquiring a relation graph, nodes in the relation graph comprising medicine nodes and protein nodes, and edges indicating that interaction exists between the nodes;
collecting, from the relation graph, a medicine node pair with definite synergism and a label of whether the medicine node pair has synergism as training samples; and
training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph attention network.

2. The method according to claim 1, wherein the relation graph is a relation graph for particular tissue and the edges in the relation graph indicate that interaction exists between the nodes on the particular tissue.

3. The method according to claim 1, wherein the medicine synergism prediction model comprises a graph attention network layer and a classification layer;
the graph attention network layer is configured to perform Attention by using feature vectors of the medicine nodes in the relation graph and feature vectors of neighbor nodes of the medicine nodes, to obtain vector representations of the medicine nodes;
the classification layer is configured to obtain, by using the vector representations of the medicine nodes in the medicine node pair, a classification result indicating whether the medicine node pair has synergism; and
a training objective of the medicine synergism prediction model is to minimize a difference between the classification result and the corresponding label.

4. The method according to claim 3, wherein initial values of the feature vectors of the nodes are obtained in the following manner:
processing the nodes by a pre-trained embedding unit to obtain feature vectors of atoms in the nodes; and integrating the feature vectors of the atoms by an integration unit, to obtain the feature vectors of the nodes as the initial values.

5. The method according to claim 4, wherein the embedding unit is pre-trained in the following manner:
acquiring a compound as a training sample; and
training a Masked Language Model (MLM) comprising an embedding unit and a prediction unit by taking training samples of some randomly masked groups as input to the MLM;
wherein the embedding unit extracts the feature vectors of the atoms in the training sample, the prediction unit predicts masked content according to the feature vectors of the atoms, and a training objective is to minimize a difference between a prediction result of the prediction unit and content of the some masked groups in the training sample.

6. The method according to claim 3, wherein the step of the performing Attention by using feature vectors of the medicine nodes in the relation graph and feature vectors of neighbor nodes of the medicine nodes, to obtain vector representations of the medicine nodes comprises:
performing multi-head attention processing by using features of the medicine nodes in the relation graph and features of the neighbor nodes of the medicine nodes; and
merging a plurality of vector representations obtained by the multi-head attention processing, to obtain the vector representations of the medicine nodes.

7. The method according to claim 3, wherein the relation graph is a relation graph for particular tissue; and
the step of obtaining, by using the vector representations of the medicine nodes in the medicine node pair, a classification result indicating whether the medicine node pair has synergism comprises:
splicing the vector representations of the medicine nodes in the medicine node pair with a vector representation of the particular tissue; and performing classification by using a vector representation obtained by splicing, to obtain the classification result indicating whether the medicine node pair has synergism.

8. The method according to claim 2, wherein the medicine synergism prediction model comprises a graph attention network layer and a classification layer;
the graph attention network layer is configured to perform Attention by using feature vectors of the medicine nodes in the relation graph and feature vectors of neighbor nodes of the medicine nodes, to obtain vector representations of the medicine nodes;
the classification layer is configured to obtain, by using the vector representations of the medicine nodes in the medicine node pair, a classification result indicating whether the medicine node pair has synergism; and
a training objective of the medicine synergism prediction model is to minimize a difference between the classification result and the corresponding label.

9. A medicine synergism prediction method, comprising:
determining a to-be-identified medicine node pair from a relation graph; and
predicting the to-be-identified medicine node pair by using a medicine synergism prediction model, to obtain a prediction result indicating whether the to-be-identified medicine node pair has synergism;
wherein the medicine synergism prediction model is pre-trained with the method according to claim 1.

10. An electronic device, comprising:
at least one processor; and
a memory communicatively connected with the at least one processor;
wherein the memory stores instructions executable by the at least one processor, and the instructions are executed by the at least one processor to enable the at least one processor to perform a method for establishing a medicine synergism prediction model, wherein the method comprises:
acquiring a relation graph, nodes in the relation graph comprising medicine nodes and protein nodes, and edges indicating that interaction exists between the nodes;
collecting, from the relation graph, a medicine node pair with definite synergism and a label of whether the medicine node pair has synergism as training samples; and
training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph attention network.

11. The electronic device according to claim 10, wherein the relation graph is a relation graph for particular tissue and the edges in the relation graph indicate that interaction exists between the nodes on the particular tissue.

12. The electronic device according to claim 10, wherein the medicine synergism prediction model comprises a graph attention network layer and a classification layer;
the graph attention network layer is configured to perform Attention by using feature vectors of the medicine nodes in the relation graph and feature vectors of neighbor nodes of the medicine nodes, to obtain vector representations of the medicine nodes;
the classification layer is configured to obtain, by using the vector representations of the medicine nodes in the medicine node pair, a classification result indicating whether the medicine node pair has synergism; and
a training objective of the model training unit is to minimize a difference between the classification result and the corresponding label.

13. The electronic device according to claim 12, wherein initial values of the feature vectors of the nodes are obtained in the following manner:
processing the nodes by a pre-trained embedding unit to obtain feature vectors of atoms in the nodes; and integrating the feature vectors of the atoms by an integration unit, to obtain the feature vectors of the nodes as the initial values.

14. The electronic device according to claim 13, wherein the embedding unit is pre-trained in the following manner:
acquiring a compound as a training sample; and
training an MLM comprising an embedding unit and a prediction unit by taking training samples of some randomly masked groups as input to the MLM;
wherein the embedding unit extracts the feature vectors of the atoms in the training sample, the prediction unit predicts masked content according to the feature vectors of the atoms, and a training objective is to minimize a difference between a prediction result of the prediction unit and content of the some masked groups in the training sample.

15. The electronic device according to claim 12, wherein the step of the performing Attention by using feature vectors of the medicine nodes in the relation graph and feature vectors of neighbor nodes of the medicine nodes, to obtain vector representations of the medicine nodes comprises:
performing multi-head attention processing by using features of the medicine nodes in the relation graph and features of the neighbor nodes of the medicine nodes; and
merging a plurality of vector representations obtained by the multi-head attention processing, to obtain the vector representations of the medicine nodes.

16. The electronic device according to claim 12, wherein the relation graph is a relation graph for particular tissue; and
the step of obtaining, by using the vector representations of the medicine nodes in the medicine node pair, a classification result indicating whether the medicine node pair has synergism comprises: splicing the vector representations of the medicine nodes in the medicine node pair with a vector representation of the particular tissue; and performing classification by using a vector representation obtained by splicing, to obtain the classification result indicating whether the medicine node pair has synergism.

17. The electronic device according to claim 11, wherein the medicine synergism prediction model comprises a graph attention network layer and a classification layer;
the graph attention network layer is configured to perform Attention by using feature vectors of the medicine nodes in the relation graph and feature vectors of neighbor nodes of the medicine nodes, to obtain vector representations of the medicine nodes;
the classification layer is configured to obtain, by using the vector representations of the medicine nodes in the medicine node pair, a classification result indicating whether the medicine node pair has synergism; and
a training objective of the model training unit is to minimize a difference between the classification result and the corresponding label.

18. A non-transitory computer readable storage medium with computer instructions stored thereon, wherein the computer instructions are used for causing a computer to perform a method for establishing a medicine synergism prediction model, wherein the method comprises:
acquiring a relation graph, nodes in the relation graph comprising medicine nodes and protein nodes, and edges indicating that interaction exists between the nodes;
collecting, from the relation graph, a medicine node pair with definite synergism and a label of whether the medicine node pair has synergism as training samples; and
training the medicine synergism prediction model by taking the medicine node pair in the training samples as input to the medicine synergism prediction model and taking the label of whether the medicine node pair has synergism as target output; wherein the medicine synergism prediction model is obtained by learning the relation graph based on a graph attention network.

19. The non-transitory computer readable storage medium according to claim 18, wherein the relation graph is a relation graph for particular tissue and the edges in the relation graph indicate that interaction exists between the nodes on the particular tissue.

20. The non-transitory computer readable storage medium according to claim 18, wherein the medicine synergism prediction model comprises a graph attention network layer and a classification layer;
the graph attention network layer is configured to perform Attention by using feature vectors of the medicine nodes in the relation graph and feature vectors of neighbor nodes of the medicine nodes, to obtain vector representations of the medicine nodes;
the classification layer is configured to obtain, by using the vector representations of the medicine nodes in the medicine node pair, a classification result indicating whether the medicine node pair has synergism; and
a training objective of the medicine synergism prediction model is to minimize a difference between the classification result and the corresponding label.
